(19) United States
(12) Patent Application Publication
(10) Pub. No.: US 2015/0374686 A1
(43) Pub. Date: Dec. 31, 2015

(54) Methods of Treating Flushing Associated with Carcinoid Tumors and Carcinoid Syndrome

(71) Applicant: Galderma S.A., Lausanne 30 Grey (CH)

(72) Inventor: Philip Manton Brown, Dallas, TX (US)

(21) Appl. No.: 14/748,391

(22) Filed: Jun. 24, 2015

Related U.S. Application Data
(60) Provisional application No. 62/019,067, filed on Jun. 30, 2014.

(51) Int. Cl. A61K 31/498; A61K 31/4164

(52) U.S. Cl. CPC A61K 31/498; A61K 31/4164

(57) ABSTRACT

A method of treating flushing associated with carcinoid tumors and carcinoid syndrome in a patient in need thereof comprising topically applying a pharmaceutical composition comprising an effective amount of an alpha adrenergic receptor agonist, pharmaceutically acceptable salt thereof, or combinations thereof; and a pharmaceutically acceptable carrier to the site of the flushing is claimed.

### CROSS-REFERENCE TO RELATED APPLICATION

This application claims priority from U.S. Provisional Application Ser. No. 62/019,067, filed Jun. 30, 2014, which is incorporated herein by reference.

### BACKGROUND OF THE INVENTION

Carcinoid tumors are rare cancerous tumors usually affecting the gastrointestinal tract or lungs. The tumors sometimes lead to the secretion of chemicals into the bloodstream causing a variety of symptoms collectively called carcinoid syndrome. About 5% to about 10% of patients with carcinoid tumors exhibit the symptoms of carcinoid syndrome. The most common symptom of carcinoid syndrome is flushing of the face, neck, and/or upper chest. Flushing includes change in color, usually redness, of the affected areas and sometimes sensations of heat.

There is a need for new therapies to treat flushing associated with carcinoid tumors and carcinoid syndrome in order to improve the quality life of patients with carcinoid tumors and carcinoid syndrome.

### SUMMARY OF THE INVENTION

The invention relates to a method of treating flushing associated with carcinoid tumors and carcinoid syndrome in a patient in need thereof by topically applying a pharmaceutical composition including an effective amount of an alpha adrenergic receptor agonist, pharmaceutically acceptable salt thereof, or combinations thereof; and a pharmaceutically acceptable carrier to the site of the flushing. In one embodiment, the site of the flushing is the face or the neck.

Preferably, the alpha adrenergic receptor agonist is an alpha-1 adrenergic receptor agonist or an alpha-2 adrenergic receptor agonist. The preferred alpha-1 adrenergic receptor agonist or alpha-2 adrenergic receptor agonist is brimonidine, tetrahydrozaline, naphazoline, xylometazoline, epinephrine, norepinephrine, oxymetazoline, phenylephrine, or methoxyamine. The most preferred alpha-2 adrenergic receptor agonist is brimonidine or a pharmaceutically acceptable salt thereof. Brimonidine or a pharmaceutically acceptable salt thereof is preferably present in an amount of from about 0.5% by weight to about 5% by weight of the composition, more preferably from about 0.5% by weight to about 2% by weight of the composition. In a preferred embodiment, the brimonidine or pharmaceutically acceptable salt thereof is present in an amount of about 0.33% by weight of the composition.

Preferably, the pharmaceutically acceptable carrier is a gel, cream, ointment, lotion, or emulsion. Gels are preferred carriers.

### DETAILED DESCRIPTION

The invention relates to a method of treating flushing associated with carcinoid tumors and carcinoid syndrome in a patient in need thereof. Carcinoid syndrome is a pattern of symptoms seen in about 5% to about 10% of patients with carcinoid tumors. The most prevalent symptom of carcinoid syndrome is flushing of the face, neck, and/or upper chest. Flushing involves the skin on the face, neck, and/or upper chest changing colors ranging from pink to red to purple and sometimes sensations of heat. Redness of the skin is most commonly associated with flushing. Flushing episodes may last a few minutes to a few hours or longer.

According to the invention, the flushing associated with carcinoid tumors and carcinoid syndrome is treated with a pharmaceutical composition including an effective amount of one or more alpha adrenergic receptor agonist or pharmaceutically acceptable salt thereof, and a pharmaceutically acceptable carrier applied topically to the skin at the site of the flushing.

Alpha adrenergic receptor agonists are well known in the art. In a preferred embodiment, the alpha adrenergic receptor agonist may be an alpha-1 or alpha-2 adrenergic receptor agonist. The alpha adrenergic receptor agonists included in the invention may or may not show selectivity for either the alpha-1 or alpha-2 adrenergic receptors. For example, some may be considered as being both alpha-1 and alpha-2 adrenergic receptor agonists. More preferably, the alpha adrenergic receptor agonist may be a selective alpha-1 or a selective alpha-2 adrenergic receptor agonist.

Examples of selective alpha-1 adrenergic receptor agonists include oxymetazoline, phenylephrine, and methoxyamine. Examples of selective alpha-2 adrenergic receptor agonists include brimonidine, tetrahydrozaline, naphazoline, xylometazoline, epinephrine, and norepinephrine.

The chemical structures of some selective alpha-1 and selective alpha-2 adrenergic receptor agonists are shown below.

| Chemical Structure | Name |
|---|---|
| 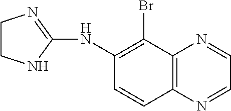 [SMILES: Brc1c(NC2=NCCN2)ccc2nccnc12] | (5-Bromo-quinoxalin-6-yl)- (4,5-dihydro-1H- imidazol-2-yl)-amine (Brimonidine) |
| 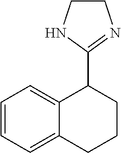 [SMILES: c1ccc2c(c1)CCCC2C1=NCCN1] | Tetrahydrozaline |
| 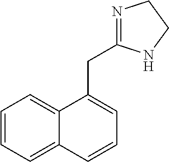 [SMILES: c1ccc2c(CC3=NCCN3)cccc2c1] | Naphazoline |
| 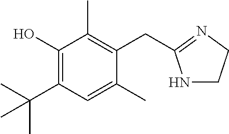 [SMILES: Cc1cc(C(C)(C)C)c(O)c(C)c1CC1=NCCN1] | Oxymetazoline |
| 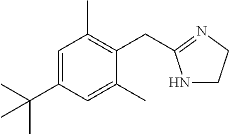 [SMILES: Cc1cc(C(C)(C)C)cc(C)c1CC1=NCCN1] | Xylometazoline |
| 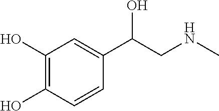 | Epinephrine |
| 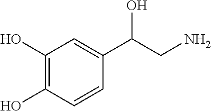 [SMILES: NCC(O)c1ccc(O)c(O)c1] | Norepinephrine |
| 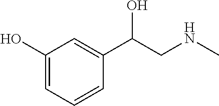 | Phenylephrine |
| 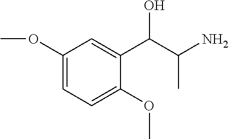 | Methoxyamine |

Brimonidine and its pharmaceutically acceptable salts are preferred embodiments of the invention. Preferably, the active ingredient of the composition is brimonidine tartrate.

The alpha-1 adrenergic receptor agonist, alpha-2 adrenergic receptor agonist, or pharmaceutically acceptable salt thereof may be administered alone or in combination with one or more alpha-1 adrenergic receptor agonist, alpha-2 adrenergic receptor agonist, or pharmaceutically acceptable salt thereof. For example, the active ingredients in the pharmaceutical composition may include brimonidine tartrate and oxymetazoline hydrochloride.

Pharmaceutically acceptable salts for each alpha adrenergic receptor agonists are well known in the art. Pharmaceutically acceptable salt means those salts of compounds of the invention that are safe and effective for topical use in mammals and that possess the desired biological activity. Pharmaceutically acceptable salts include salts of acidic or basic groups present in compounds of the invention. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the invention can form pharmaceutically acceptable salts with various amino acids. Suitable base salts include, but are not limited to, aluminum, calcium, lithium, magnesium, potassium, sodium, zinc, and diethanolamine salts. Pharmaceutically acceptable salts are discussed in BERGE ET AL., 66 J. PHARM. SCI. 1-19 (1977), incorporated herein by reference.

Pharmaceutical compositions include any formulations which are pharmaceutically acceptable for topical delivery of the compounds of the invention. The choice of topical formulation will depend on several factors, including the physiochemical characteristics of the particular compound(s) of the invention and of other excipients present, their stability in the formulation, available manufacturing equipment, and cost constraints.

The pharmaceutically acceptable composition is applied locally to the site of the affected skin of the patient in any conventional manner well known in the art. For example, the composition may be a gel that can be applied to the face and neck of the patient using the fingertips.

The amount of alpha adrenergic receptor agonist applied to the skin is any amount that is effective in treating flushing associated with carcinoid tumors and carcinoid syndrome. Generally the minimum amount of an alpha adrenergic receptor agonist in a topical formulation of the invention applied to the affected skin area is about 0.0001 g/cm², preferably about 0.001 g/cm² of skin surface area. The maximum amount of an alpha adrenergic receptor agonist in a topical formulation of the invention applied to the affected skin area is about 0.05 g/cm² to about 0.008 g/cm² of skin surface area. Typically, one to four applications per day are recommended during the term of treatment.

Dosages and dosing frequency will be determined by a trained medical professional depending on the activity of the compound of the invention, the characteristics of the particular topical formulation, and the general physical condition of the person being treated.

In general, each alpha adrenergic receptor agonist or pharmaceutically acceptable salt thereof is present in a formulation of the invention in a minimum amount of from about 0.05 percent, about 0.1 percent, or about 0.15 percent of the total weight of the formulation. Preferably, an alpha adrenergic receptor agonist or pharmaceutically acceptable salt thereof is present in a formulation of the invention in a maximum amount of about 5 percent, about 2 percent, about 1 percent, or about 0.5 percent of the total weight of the formulation.

It is to be understood that the present invention contemplates embodiments in which each minima is combined with each maxima to create all feasible ranges. For example, each alpha adrenergic receptor agonist or pharmaceutically acceptable salt thereof may be present in a composition of the invention in an amount of from about 0.05 percent to about 1 percent based upon the total weight of the composition or likewise from about 0.1 percent to about 1 percent based upon the total weight of the composition.

In one embodiment, the compounds of the invention are delivered to the affected area of the skin in a pharmaceutically acceptable carrier. As used herein, a pharmaceutically acceptable carrier is any pharmaceutically acceptable formulation that can be applied to the skin surface for topical or dermal delivery of a pharmaceutical or medicament. The combination of a pharmaceutically acceptable carrier and a compound of the invention is termed a pharmaceutical composition of the invention. Pharmaceutical compositions of the invention are prepared by mixing a compound of the invention with a topical carrier according to well-known methods in the art, for example, methods provided by standard reference texts such as, REMINGTON: THE SCIENCE AND PRACTICE OF PHARMACY 1577-1591, 1672-1673, 866-885 (Alfonso R. Gennaro ed. 19th ed. 1995); Ghosh, T. K.; et al. TRANSDERMAL AND TOPICAL DRUG DELIVERY SYSTEMS (1997), both of which are hereby incorporated herein by reference. The discussion of pharmaceutical compositions containing alpha adrenergic receptor agonists from U.S. Pat. No. 7,439,241 is incorporated herein by reference.

The topical carriers useful for topical delivery of compounds of the invention can be any carrier known in the art for topically administering pharmaceuticals, for example, but not limited to, pharmaceutically acceptable solvents, such as a polyalcohol or water; emulsions (either oil-in-water or water-in-oil emulsions), such as creams or lotions; micro emulsions; gels; ointments; liposomes; powders; and aqueous solutions or suspensions. The preferred carriers are gels, creams, ointments, lotions, and emulsions.

### EXAMPLES

### Example 1

### Gel Composition

| Ingredient | Weight Percent |
|---|---|
| Brimonidine tartrate | 0.33% |
| Carbomer 934P | 1.25% |
| Methylparaben | 0.3% |
| Phenoxyethanol | 0.4% |
| Glycerin | 5.5% |
| 10% Titanium dioxide | 0.625% |
| Propylene glycol | 5.5% |
| 10% NaOH Solution | 6.5% |
| DI Water | QS |
| TOTAL | 100% |

### Example 2

### Cream Composition

| Ingredient | Weight Percent |
|---|---|
| Brimonidine tartrate | 0.5% |
| Oxymetazoline hydrochloride | 0.5% |
| Phenoxyethanol | 0.8% |
| Methylparaben | 0.2% |
| Propylparaben | 0.05% |
| Disodium EDTA | 0.01% |
| Butylated Hydroxytoluene | 0.05% |
| PEG-300 | 4.0% |
| PEG-6 Stearate (and) Glycol | 7.5% |
| Stearate (and) PEG-32 Stearate | |
| Cetostearyl alcohol | 4.0% |
| Caprylic capric triglycerides | 7.0% |
| Diisopropyl adipate | 7.0% |
| Oleyl alcohol | 7.0% |
| Lanolin USP | 2.0% |
| Ceteareth-6 (and) Stearyl | 2.0% |
| Alcohol | |
| Ceteareth-25 | 2.0% |
| Tartaric Acid | 0.001% |
| DI Water | 55.389% |
| TOTAL | 100% |

### Example 3

### Lotion Composition

| Ingredient | Weight Percent |
|---|---|
| Brimonidine tartrate | 0.25% |
| Amerlate P Isopropyl Lanolate | 0.5% |
| Stearic Acid | 3.0% |
| Glyceryl Stearate | 2.0% |
| Methyl Gluceth-20 | 5.0% |
| Triethanolamine | 1.0% |
| Water | 83.45% |
| Polyquaternium-24 and | 3.8% |
| Hyaluronic Acid (BIOCARE | |
| Polymer HA-24) | |
| Germaben IIE | 1.0% |
| TOTAL | 100% |

### Example 4

A gel containing 0.33 weight % brimonidine tartrate is administered to the face and neck of a patient with carcinoid syndrome. The patient applies the gel to his skin twice a day as necessary.

We claim:

1. A method of treating flushing associated with carcinoid tumors and carcinoid syndrome in a patient in need thereof comprising topically applying a pharmaceutical composition comprising an effective amount of an alpha adrenergic receptor agonist, pharmaceutically acceptable salt thereof, or combinations thereof; and a pharmaceutically acceptable carrier to the site of the flushing.

2. A method according to claim 1, where the site of the flushing is the face or the neck.

3. A method according to claim 1, wherein the alpha adrenergic receptor agonist is an alpha-1 adrenergic receptor agonist or an alpha-2 adrenergic receptor agonist.

4. A method according to claim 1, wherein the alpha-1 adrenergic receptor agonist or alpha-2 adrenergic receptor agonist is brimonidine, tetrahydrozaline, naphazoline, xylometazoline, epinephrine, norepinephrine, oxymetazoline, phenylephrine, or methoxyamine.

5. A method according to claim 1, wherein the alpha-2 adrenergic receptor agonist is brimonidine or pharmaceutically acceptable salts thereof.

6. A method according to claim 1, wherein the pharmaceutically acceptable carrier is a gel, cream, ointment, lotion, or emulsion.

7. A method according to claim 1, wherein the brimonidine or pharmaceutically acceptable salt thereof is present in an amount of from about 0.5% by weight to about 5% by weight of the composition.

8. A method according to claim 1, wherein the brimonidine or pharmaceutically acceptable salt thereof is present in an amount of from about 0.5% by weight to about 2% by weight of the composition.

9. A method according to claim 1, wherein the carrier is a gel and the brimonidine or pharmaceutically acceptable salt thereof is present in an amount of about 0.33% by weight of the gel.
